(19) United States
(12) Patent Application Publication
(10) Pub. No.: US 2007/0088376 A1
(43) Pub. Date: Apr. 19, 2007

(54) Precision surgical system

(76) Inventor: JAIME ZACHARIAS, Santiago (CL)

Correspondence Address: LAW OFFICE OF JAY R. YABLON, 910 NORTHUMBERLAND DRIVE, SCHENECTADY, NY, 12309-2814, US

(21) Appl. No.: 11/164,164

(22) Filed: Nov. 13, 2005

Related U.S. Application Data
(60) Provisional application No. 60/596,740, filed on Oct. 18, 2005.

(51) Int. Cl. A61B 17/32

(52) U.S. Cl. 606/169

(57) ABSTRACT

A high-speed surgical handpiece (10) suitable for vitreoretinal surgery having a cutter (42) and actuators (36). The cutter (42) is a guillotine-type cutter activated by an array of leveraged piezoelectric actuators (30) that receive a driving signal from a driving controller. The controller can have control and display units with a plurality of input mechanisms receiving input from a user who selects a desired cutting rate and frequency for the cutter. The control unit produces a piezoelectric actuator output signal based on the inputs received. Fast cutting rates with reduced duty cycle as well as a proportional mode of operation are available, allowing slow controlled cutting action, for example proportional to depression of a foot-pedal (74). Low degrees of vibration and noise generation are produced.

### CROSS REFERENCES TO RELATED APPLICATIONS

This application claims the benefit of related U.S. Provisional Patent Application No. 60/596740 filed Oct. 18, 2005 titled “Piezoelectric Vitrectomy Probe”.

### FIELD OF THE INVENTION

This invention is related to electrically operated surgical systems, and more particularly to a surgical system of the kind suitable for vitreoretinal surgery powered by a piezoelectric mechanism.

### DISCUSSION OF PRIOR ART

The intraocular portion of current vitrectomy probes typically consists in a closed end outer tube having a distal end sideport to aspirate the vitreous, and an inner tube that oscillates axially during operation in a way that the distal end sharp edge can displace with a cutting action across said sideport. Oscillation of the inner tube is typically provided by pneumatic turbines and electric rotary motors. Also, diaphragm based pneumatic systems have been used operated by fast changes in pressure levels inside a gas chamber at the handpiece proximal portion.

These changes in pressure levels are console driven typically consisting in the alternation of positive and negative pressure cycles at the operation frequency desired for the cutter. Vacuum applied by a vacuum source in fluid communication with the hollow oscillating tube aspirates the vitreous into the sideport and the axially oscillating inner tube distal end sharp edges cut the vitreous allowing aspiration and removal of the vitreous and any other intraocular material to be removed.

A fluid source in direct communication with the intraocular cavity can provide pressurized balanced salt solution to replace the volume of the removed vitreous. There would be are advantage in increasing the speed of operation of vitrectomy cutters as less traction would be applied to the vitreous body and the displacement of tissue into the aspirating sideport would be more controlled and continuous. Currently available pneumatic vitreous cutters can operate up to 1.500 cuts per minute but typically exhibit a reduced duty cycle.

Electrically driven vitreous cutters can operate at higher speeds, up to 3.000 cuts per minute, but are typically heavy, delicate and vibrate during operation. These details have been exposed in U.S. Pat. No. 6,575,990 the one I incorporate here as a reference. U.S. Pat. No. 6,875,221 also cited here with its accompanying references to provide background for the present description.

Typically, the speed of the cutting blade of currently available electrically operated vitrectomy handpieces is proportional to the cut rate. When operating at low cut rates, the blade traverses the cutting sideport at a lower speed than when operating at higher cutting rates. This mode of operation is related to the rotary coupled mechanism of many electric vitrectomy handpieces. Pneumatic handpieces exhibit a progressive increase duty cycle portion where the sideport is closed as the cut rate is increased, as physical limitations apply to recycle the guillotine cutter with its biasing preloading spring.

One limitation of known vitreous cutters operating at high speed is that the duty cycle portion where the sideport is open becomes progressively reduced as the operating speed is increased. This increase of the portion where the sideport is closed with respect to the duration of one full cycle reduces cutter efficiency as less time is available for vacuum to aspirate vitreous tissue into the sideport for the cutting and aspirating action. The reduced efficiency increases surgical time increasing complications such as post-vitrectomy cataract formation and reduces operating room turn around.

Another limitation of current vitrectomy cutters operating at high speed is that there is vibration of the tip related to movements of the internal mechanisms used to power the cutting edges. Another limitation of current vitrectomy cutters is that regulation of the open sideport area cannot be adjusted or requires manual mechanical adjustments at handpiece level. Still another limitation of current vitrectomy cutters operated at high speed is that the vibration of the internal mechanisms used to power the cutting edges produces noise.

Still another limitation of current vitrectomy cutters is that they do not allow direct control of the cutting blade following an analog footpedal command that produces a displacement of the blade proportional to or as a function of the displacement of the footpedal or other analog user interface input. Still another limitation of current electric vitrectomy cutters is that speed of the cutting blade is coupled to the cutting rate. Still another limitation in the case of pneumatic cutters is that the effective open sideport ratio is reduced as the cut rate is increased near the upper end.

There is still a need for vitrectomy cutters that can operate in the high speed range to cut the vitreous as well as at very low speeds including direct analog control to operate forceps and scissors in proportional mode. Also, there is a need for vitrectomy cutters providing maximum sideport open ratios preferably above 50% when operating at cut rates 1.500 cuts per minute. Also, there is a need for vitrectomy cutters that operate the cutting blade at a speed that is independent of the cut rate, in a way that the blade traverses the cutting sideport at high speed even when operating at lower cut rates to improve efficiency and duty cycle.

Also, there is a need for vitrectomy cutters that allows an operator to adjust the area of the open sideport console user interface level. Also there is the need for a high speed vitreous cutting handpieces that is lightweight, operate silently and produce a minimum of vibration. Also there is the need for a high speed vitreous cutting handpieces that is mechanically simple allowing repeated sterilization and providing reduced wear and failure rates.

### OBJECTS AND ADVANTAGES

It is an object of the present invention to provide a vitreous cutter mechanism that allows a fast cutting speed of the cutting edge across the aspirating sideport irrespective of the cutting rate in cuts per minute. It is another object of the present invention to provide a vitrectomy probe that can operate efficiently at speeds above 2.000 cuts per minute. It is still another object of the present invention to provide a vitreous cutter handpiece where the open sideport ratio is above 50% at high operating frequencies.

It is still another object of the present invention to provide a vitreous cutter handpiece that allows adjustment of the position of the cutting border within a vitrectomy handpiece cutting sideport to regulate the effective area of the open sideport. It is still another object of the present invention to provide a vitreous cutter handpiece that also allows an operator to displace the cutting border across a vitrectomy sideport following a footpedal command or other proportional user interface inputs.

It is still another object of the present invention to provide a vitreous cutter handpiece that operates silently and that produces a minimum of actuator-related vibration during operation. It is still another object of the present invention to provide a vitreous cutter handpiece that is lightweight and resistant to sterilization. It is still another object of the present invention to provide a vitreous cutter mechanism that is mechanically simple with reduced wear and failure rates.

### BRIEF DESCRIPTION OF THE DRAWINGS

FIG. 1 depicts a schematic view of a vitrectomy system incorporating the handpiece of the present invention.

FIG. 2 depicts a schematic external view of the vitrectomy handpiece.

FIGS. 3A and 3B respectively depict an illustration of an amplified piezoelectric actuator in contracted and expanded position.

FIG. 4 is an overall view of a handpiece of the present invention with a removed portion of the enclosure to allow visualization of the stack of amplified piezoelectric actuators that powers the cutting mechanism.

FIG. 5 is a schematic lateral view of the handpiece of the present invention with the amplified piezoelectric actuators in contracted position and consequently with the guillotine in open position.

FIG. 6 is a schematic lateral view of the handpiece of the present invention with the amplified piezoelectric actuators in expanded position and consequently with the guillotine in closed position.

FIG. 7 is a schematic lateral view of an alternative handpiece of the present invention that incorporates a vibration detection mechanism and a vibration canceling mechanism with the guillotine in open position.

FIG. 8 is a schematic lateral view of an alternative handpiece of the present invention that incorporates a vibration detection mechanism and a vibration canceling mechanism with the guillotine in closed position.

FIG. 9A is a schematic lateral view of an alternative handpiece of the present invention incorporating a telescopic piezoelectric actuator shown here in contracted position and consequently with the guillotine in open position.

FIG. 9B is a mid cross sectional view of the handpiece depicted in FIG. 9A showing the concentric array of interleaved expanding and contracting piezoelectric elements.

FIG. 10 is a schematic lateral view of an alternative handpiece of the present invention incorporating a telescopic piezoelectric actuator shown here in expanded position and consequently with the guillotine in closed position.

FIG. 11 is a detailed schematic lateral view of the rear portion of a handpiece of the present invention operated using a telescopic piezoelectric actuator and showing the reverse polarity connection of the odd and even piezoelectric tubes.

FIG. 12 is a schematic lateral view of an alternative handpiece of the present invention incorporating a stack of bimorph disk piezoelectric actuators.

FIG. 13 is a schematic diagram of a vitrectomy system incorporating the handpiece of the present invention.

### LIST OF REFERENCE NUMERALS

Surgical handpiece 10, vitrectomy prove proximal end 11, vitrectomy probe 12, vitrectomy probe distal end 13, vitrectomy probe sideport 14, guillotine cutting edge 15, surgical handpiece body 16, detachable head 17, aspiration port 18, aspiration tubing 19, body-head coupling 29, surgical handpiece cable 20, actuator driver cable 21, amplified piezoelectric actuator 30, actuator connection pad 32, amplified piezoelectric actuator leveraging fame 34, piezoelectric actuator 36, interlock coupling 40, aspiration duct 42, guillotine 44, load spring 48, external expanding tube 60, central expanding tube 62, internal expanding tube 64, surgical system console 70, user interface 71, controls 72, display 73, footpedal 74, footpedal cable 75, footpedal connector 76, aspiration tubing connector 77, surgical handpiece cable connector 78, position sensor 80, position sensor cable 81, vibration sensor 82, vibration sensor cable 83, vibration canceling actuator 84, vibration canceling actuator cable 85, vibration canceling mass 86, pressurized balanced salt solution 90, solenoid 92, infusion tubing 94, eye 96, irrigation incision 97, vitrectomy probe incision 98, actuator fixation 100, bimorph disk actuator 130, external contracting tube 170, internal contracting tube 172, opposed amplified piezoelectric actuator faces 210 and 212.

### SUMMARY

An electrically powered vitrectomy handpiece operated by the action of leveraged piezoelectric actuators allowing an improved range of speeds of operation from direct control to above 1.500 cuts per minute, high guillotine displacement speed, improved sideport open ratio characteristics and reduced vibration and noise generation.

### DESCRIPTION OF THE PREFERRED EMBODIMENT

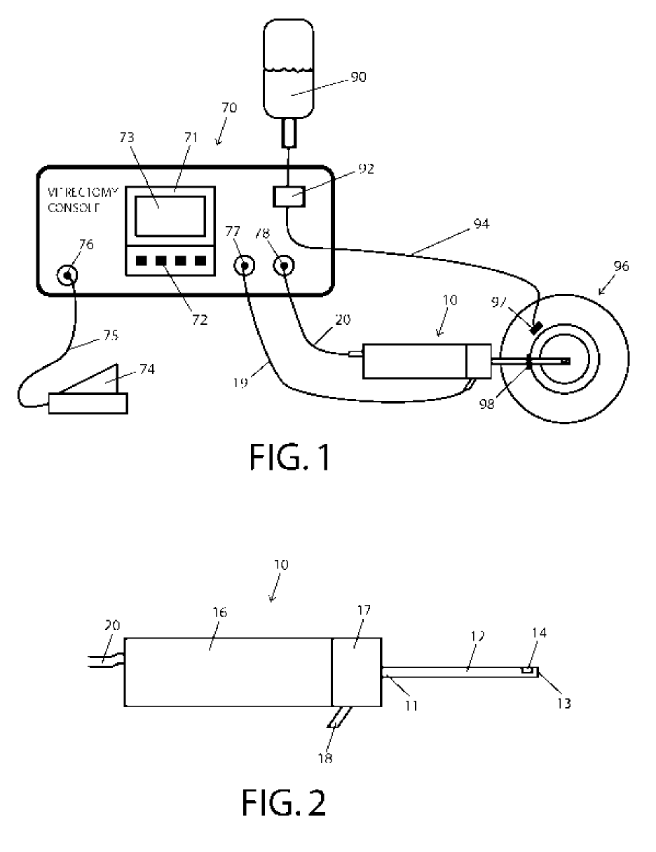

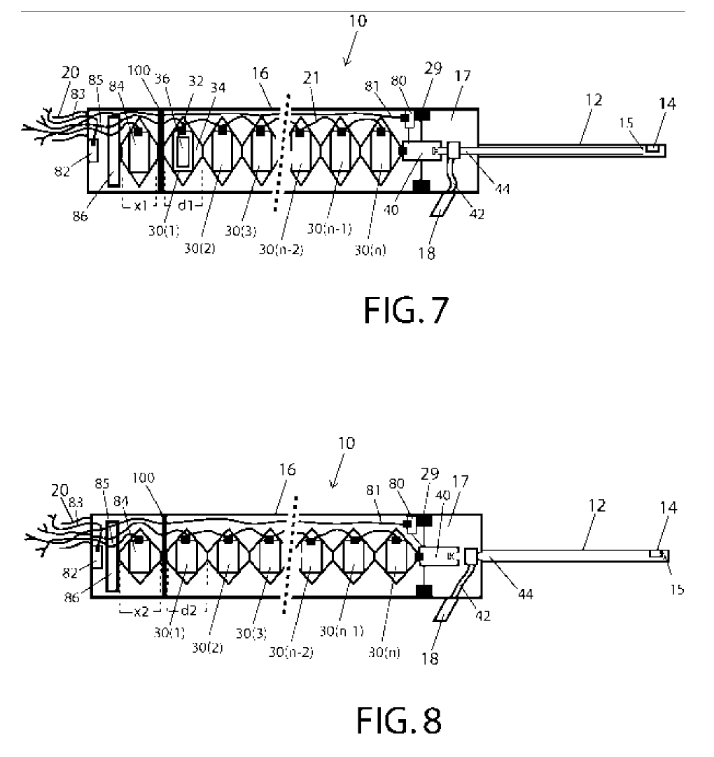

A vitrectomy system incorporating a vitrectomy handpiece 10 of the present invention as shown in FIGS. 1 and 7 is composed of a vitrectomy console 70 including a user interface 71 with operator controls 72 and a display 73. A source of pressurized balanced salt solution 90 can be delivered into an eye 96 through an infusion tubing 94 placed across a solenoid 92 and into an irrigation incision 97 of an eye 96. A footpedal 74 is connected to console 70 through a cable 75 and a connector 76. Console 70 can also provide to vitrectomy handpiece 10 a source of vacuum through a connector 77 and an aspiration tubing 19 inserted into an aspiration port 18, with vitrectomy handpiece 10 eventually inserted into eye 96 through a vitrectomy incision 98.

A connector 78 provides electric communication between console 70 across electric conductor cable 20 with actuator 30, 34 and sensor elements 80, 82 inside a body 16 of handpiece 10. Referring now to FIGS. 1 and 2, handpiece 10 of the present invention is composed of a body 16 and a detachable head 17. Detachable head 17 includes a hollow vitrectomy probe 12 having a proximal end 11 and a distal end 13.

A vitrectomy sideport 14 is preferably located near vitrectomy probe 12 distal end 13. Aspiration port 18 is in fluid communication with sideport 14 through a tubing 42. Aspiration port 18 can connect through aspiration tubing 19 and connector 77 with an aspiration source provided by vitrectomy console 70. The vitreous cutting mechanism of handpiece 10 of the present invention is activated by the action of piezoelectric electro-mechanic actuators. It is known fact that typical single element or stack based piezoelectric actuators provide high force but limited displacement.

The guillotine cutter of a vitrectomy handpiece will require a stroke above 400 microns to fully displace across a typical vitrectomy sideport. This stroke cannot be achieved with the required force using piezoelectric actuators in a typical configuration within the practical dimensions and weight of a standard vitrectomy handpiece. This invention is based on the use of one or more leveraged piezoelectric actuators preferably in the form of amplified piezoelectric actuators or telescopic piezoelectric actuators to activate a vitrectomy handpiece.

Externally, internally and telescopic leveraged piezoelectric actuators can be considered. Between the different geometric configurations, externally leveraged piezoelectric actuators, preferably in the form of amplified piezoelectric actuators such as Cedrat APA50XS can be used with advantage in this application(Cedrat Technologies, 15 Chemin de Malacher, ZIRST, 38246 Meylan Cedex, France, http://www.cedrat.com). Also, piezoelectric actuators based on single or stacked telescopic architectures or serially coupled disk translators, such as P-288 HVPZT provided by Physik Instrumente can be used. Each of these architectures has its characteristic static, quasi-static and dynamic properties and can be used in different embodiments of this invention.

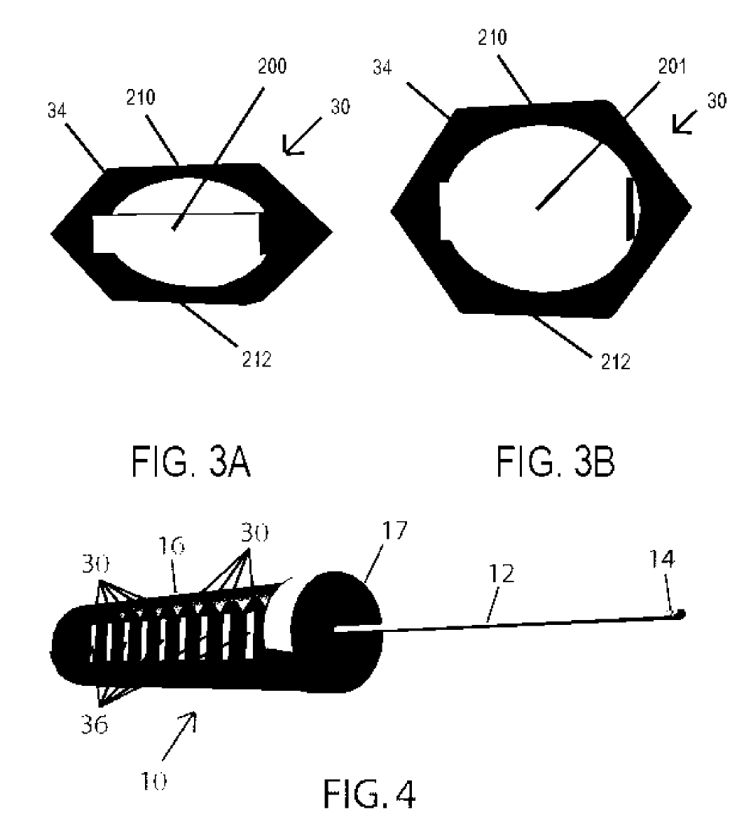

Externally leveraged piezoelectric actuators increase the stroke of piezoelectric elements at the expense of force by using geometric configurations that multiply stroke of the piezoelectric elements in the range of 20X. As considered for the preferred embodiment of the present invention, an amplified piezoelectric actuator 30 shown in FIGS. 3A and 3B consists in a piezoelectric element 36 inserted within a pre-tensed elastic frame 34. The geometry of frame 34 determines that the expansion of piezoelectric element 36 by the action of a voltage differential produces an amplified contraction at an axis that is perpendicular to the main axis of the mechanical deformation of piezoelectric element 36 and parallel to the plane of frame 34, mainly between faces 210 and 212.

This contraction is produced by the elastic properties of frame 34 deformed by the expansion of piezoelectric element 36. In reverse, contraction of piezoelectric element 36 produces an amplified expansion between faces 210 and 212. Contraction and expansion are said to be amplified because the magnitude of displacement between faces 210 and 212 is higher than the magnitude of displacement of the activating piezoelectric element 36.

FIG. 3A depicts a typical configuration for an amplified piezoelectric actuator 30 in the contracted mode. Piezoelectric element 36 is expanded by the action of a driving voltage deforming elastic frame 34 in a way that parallel surfaces 210 and 212 approximate in an extent bigger than the longitudinal expansion of the perpendicular piezoelectric element 36.

FIG. 3B depicts the amplified piezoelectric actuator 30 in the opposite expanded mode. Here piezoelectric element 36 is contracted by the action of a driving voltage of a reverse polarity with pre-stressed elastic frame 34 following the contraction of piezoelectric element 36 in a way that parallel surfaces 210 and 212 separate in an extent bigger than the longitudinal contraction of the driving piezoelectric element 36.

A preferred embodiment of the surgical handpiece 10 of the present invention is shown in FIG. 4. Here an array of single amplified piezoelectric actuators 30 is mechanically connected in series and axially disposed to activate a guillotine type vitrectomy cutter, scissors or forceps. Each suitably sized commercially available amplified piezoelectric actuator (APA50XS) to be used in this invention can provide an axial displacement of 80 microns.

As a mode of example, a stack of ten amplified piezoelectric actuators APA50XS mechanically coupled in series and powered simultaneously can provide a total axial displacement for a vitrectomy cutter, scissors or forceps of 800 microns at diverse speeds ranging from direct control (DC) to above 2.000 cuts per minute. Force using this kind of amplified piezoelectric actuators can extend up to 15 Newton, enough to power a typical guillotine type vitrectomy cutter.

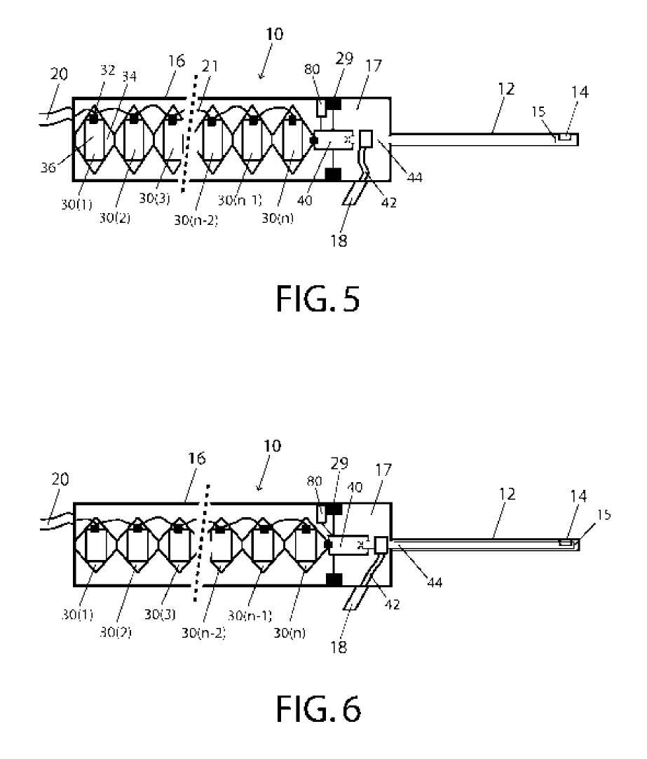

As depicted in FIGS. 5 to 10, detachable head 17 includes hollow vitrectomy probe 12 with an internally disposed guillotine cutter 44 with a cutting border 15 sliding with a cutting action across the inner aspect of sideport 12. When not occluded by guillotine cutter 44, sideport 12 is in fluid communication with aspiration port 18 through an aspiration channel inside hollow vitrectomy needle 12, and fluid connector 42. Aspiration port 18 can be connected to a vacuum source typically provided by vitrectomy console 70.

Hollow vitrectomy needle 12, guillotine 44, aspiration port 18 and vacuum connector 42 are incorporated into handpiece head 17 that can be detachably connected to operate in conjunction with handpiece body 16. Head 17 is detachably connected using an attachment mechanism 19 preferably based on a bayonet or threaded coupling.

In a preferred embodiment shown in FIGS. 5 and 6, enclosed within handpiece body 16 is a plurality of amplified piezoelectric actuators 30. Each amplified piezoelectric actuator 30 is composed of a of single or stacked piezoelectric elements 36 perpendicularly disposed inside pre-stressed frames 34 and connected to a piezoelectric driver circuit inside console 70 through cable 21, cable 20 and connector 78.

A suitable selection for an amplified piezoelectric actuator for a vitrectomy handpiece 10 of the present invention can be similar to APA50XS this model having a height of 4.7 mm, a width of 9.0 mm and a depth of 12.8 mm. As a mode of example, a stack of ten APA50XS will have a height of 47 mm, a width of 9 mm and a depth of 12.8 mm, dimensions compatible with a typical surgical handpiece for vitrectomy having a radius of 8 mm and weight below 30 grams.

Customization of the dimensions of individual amplified piezoelectric actuators 30 can provide several other alternative configurations to adapt to different handpiece requirements including static, quasi-static and dynamic properties, dimension and weight. As shown in FIG. 8 amplified piezoelectric actuators 30(1) to 30(n) are mechanically connected in series and can be electrically connected in a parallel configuration through electric cable 21 attached to pads 32. The blocked end of the full array of amplified piezoelectric actuators array is fixed to fixed structure of handpiece body 16.

The opposite free end of the array of amplified piezoelectric actuators 30 is mechanically coupled to a detachable interlocking connector 40 capable of mechanically coupling with guillotine cutter 44 in a push-pull configuration when handpiece head 17 is placed in operation position. Coupling between interlocking mechanical connector 40 and guillotine shaft 44 is designed using known methods to provide minimum backlash preferably below 5 microns while still being detachable for head sterilization and exchange. The total number of amplified piezoelectric actuators 30(1) to 30(n), where n is the total number of actuators in the array will vary according to the maximum stroke required to drive a particular vitrectomy cutter, scissors or forceps. A position sensor element 80 can be disposed inside handpiece body 16 to detect the axial position of the piezoelectric actuators array during operation.

The position sensor element 80 can be constituted by one or more strain gauges, capacitive position sensors, optical position sensors, LVDTs or any other position sensor elements suitable to detect in real time the axial position and displacement information of the free movable end of the array of actuators 30(1) to 30(n).

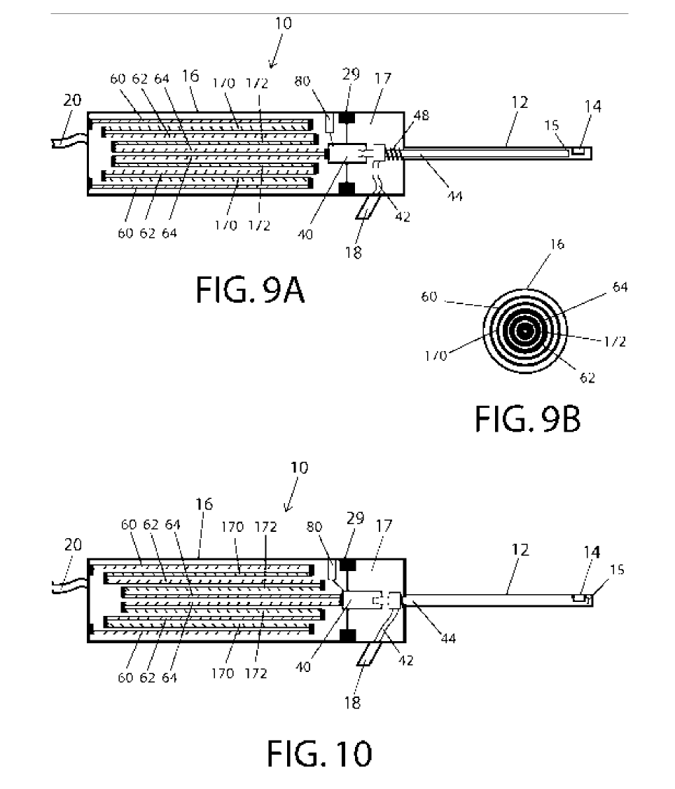

Position sensor element 80 connects to console 70 sequentially through cables 81, 20 and connector 78. As shown in FIG. 9A a preloading spring 48 can be disposed in the mechanical path between the piezoelectric actuators 30 and the cutting border 15.

### OPERATION OF THE PREFERRED EMBODIMENT

During operation, an operator holds handpiece 10 by its body 16 and the hollow vitrectomy needle 12 can be inserted into an eye 96 through an incision 98. An aspiration source can be connected to port 18 in fluid communication with cutting port 14. Irrigation solution can be provided to the interior of eye 96 through an irrigation line 94 using an irrigation incision 97. Following an operator commands a suitable electrical signal is provided by vitrectomy console 70 through cables 20 and 21, the voltage typically ranging between −20 and +150 volts. According to the piezoelectric effect, a varying voltage level will make the piezoelectric elements 36 inside each amplified piezoelectric actuators 30 to expand or contract altering the geometry of the surrounding elastic frames 34 in a way that a linear multiplication of the deformation of the piezoelectric elements occurs.

This deformation amplifies in inverting configuration the expansion and contractions of the piezoelectric elements 36 perpendicularly and at the plane of frames 34. FIGS. 7 and 8 illustrate this dimensional change showing axial distance d1 increasing to distance d2 between parallel surfaces 210 and 212 by operation of actuator 30. Each amplified piezoelectric actuator 30 of the preferred kind suitable for this application can typically displace 80 microns. As a mode of example, a total of ten amplified piezoelectric actuators mechanically coupled in series as shown in FIGS. 5 to 8 can produce a linear displacement of 800 microns when driven with voltage levels ranging between −20 and +150 volts.

The maximum force exerted can reach up to 15 Newton with an unloaded response time bellow 2 milliseconds. The summed displacement and force of the amplified piezoelectric actuators is transmitted across interlocking connector 40 to guillotine cutter 44. Guillotine cutter 44 cutting border 15 displaces an amount equivalent to the summed action of amplified piezoelectric actuators 30(1) to 30(n) and is pre-adjusted to internally travel across the opening of sideport 14 exerting a cutting action. According to operator settings at console 70 level, different cutting border 15 axial displacement patterns can be obtained. As a mode of example, maximum cutting rates above 2.000 cuts per minute can be obtained with an open-to-close ratio of cutting sideport 14 above 3 (open duty cycle above 75%).

The waveform of the linear axial displacement of cutting border 15 can be adjusted according to user preferences, for example between sinusoidal, square, saw-tooth and others. Regulation of the maximum effective open size of aspiration port 14 can be performed at console 70 level by providing a biasing direct current (DC) level to partially displace the cutting border 15 across sideport 14 when in open position, to reduce the open size of sideport 14 to reduce the chances of intraocular structure damage when operating the vitrectomy needle near sensitive tissues such as the retina. A direct control cutting action adequate to aspirate and cut minute portions of tissue near delicate structures can be obtained by replacing a driving alternating frequency by a driving footpedal controlled direct current (DC) level.

In this way, an operator can open and close the aspiration sideport 14 and cut tissues with a displacement of cutting border 15 that is proportional to or a function of analog footpedal 74 activation. Position sensor 80 detects in real time the position of the movable end of the array of actuators 30(1) to 30(n) that also corresponds to the position of guillotine 44 inside needle 12. Static and dynamic position information provided by position sensor 80 is processed at console level to adjust operating conditions depending on particular characteristics of the head 17, for example adjusting the guillotine 44 open and closed position according to different sideport 14 location and size. In a typical configuration, position sensor 80 sends a position sensor signal to console 70 where a position sensor module conditions the position sensor signal and feeds it to a handpiece controller system including a microprocessor or digital signal processor (DSP).

Position signal information can be digitized preferably at a sampling rate above 1000 hertz. Microcontroller or DSP unit can use the position sensor information acquired at high speed to dynamically adjust the amplified piezoelectric actuators 30 driving signal provided by a cutter actuator module determining a feedback for closed loop, position servo control system. These adjustments can be programmed at microcontroller or DSP level to keep the desired stroke constant during operation irrespective of temperature or other drift creating conditions. In this way hysteresis and creep behaviors associated with slow realignment of the crystal domains in a constant electric field can be controlled.

Also, the feedback signal provided by position sensor 80 can also be used to implement a vibration canceling algorithm. A typical resonant frequency for the handpiece of this invention is about 333 hertz being sensitive to total mass variation and cables location. Resonant frequency fo for a piezoelectric system is a function of actuators stiffness Kt and total mass Mt., being Mt the sum of the effective mass Meff plus any mass attached at the end piece Mep. According to the formula to determine fo, the resonant frequency will drop by a factor of 2 when the total mass is increased by a factor of 4. It is known that a piezoelectric element will have a settling time typically equivalent to ⅓ of the duration of one period of the resonant frequency. In the constructed prototype of this invention, ⅓ of one period ( 1/333 Hz) equals 0.001 second, in response to a step change in voltage using a driver with sufficient current output and short rise time.

Step responses of piezoelectric actuators typically have overshoot and structural ringing. By incorporating position sensor 80 that provides feedback to the driver circuit, a reduction of undesired nonlinearities, drift, overshoot and structural ringing is achieved. In a closed loop servo-controller configuration, the feedback position signal provided by sensor 80 is processed at the piezoelectric driver module and/or at microcontroller/DSP level to adjust the magnitude of the output signal produced by the driver module to obtain the desired displacement.

A typical servo-controller system for this application determines the output voltage to the piezoelectric elements comparing a reference signal to achieve the commanded position with the actual position informed by the position sensor 80. Using simple servo-controller designs provides a closed loop tracking bandwidth up to 1/10^{th} of the resonant frequency. When the surgical system of the present invention is used near the upper frequency limit and to keep the open-to-closed sideport ratio (duty cycle) to a minimum, it is considered to use more sophisticated methods to improve system dynamics.

The use of a resonance canceling algorithm that operates based on a predictive model can avoid ringing and mechanical resonances in both step-mode and repetitive mode operation of the surgical handpiece of the present invention. An implementation of the patented Input-Shaping™ method can be used with advantage to increase bandwidth in this surgical system (http://www.convolve.com). Also, another ring canceling method known as signal pre-shaping can be used in repetitive modes of operation. The method reduces roll-off, phase error and hysteresis of the servo system using FFT techniques at microcontroller or DSP level.

Frequency response and harmonics are detected and corrections are applied to the control function of the piezoelectric elements driver in an iterative process until unwanted motion patterns are cancelled out. Using these methods, the bandwidth of proper operation of the servo-control system can be expanded in the high frequency side in one order of magnitude. Also, actuator compensation information can be stored in ROM during manufacture and testing, and implemented according to the selected operator settings.

In a typical configuration, operation of the system can consider a first stage of tests and a second stage of actual surgical use. The testing process can consider determining the resonant frequency of the handpiece body alone to check that the piezoelectric elements and the whole mechanical system is in good condition. Deviation of the resonant frequency from an expected range can be a signal of handpiece damage. Voltage/current (V/I) phase determination can complement the resonant frequency test.

In systems that incorporate a position sensor element 80 the desired versus actual displacement of the actuator system can be measured to check for proper operating conditions. These tests can be performed with detachable head 17 placed in operating conditions to check for proper detachable head status. During use of the surgical system of the present invention, an operator selects via user interface 71 the operation modality of footpedal 74. One mode of operation is proportional and the other is repetitive.

Proportional Mode:

In proportional mode, the piezoelectric driver module provides a variable DC output voltage that is proportional to the degree of displacement of the footpedal 74 or other analog user interface input. In this mode handpiece 10 can perform single controlled activation with a vitrectomy, scissors or forceps surgical instrument installed. Typically an output voltage proportional to footpedal 74 position is produced at piezoelectric driver module and delivered through connector 78, cables 20 and 21 to piezoelectric elements 36.

Voltage variation is configured to produce a contraction of piezoelectric elements 76 in a way that elastic frames 34 are deformed producing an increase in distance between surfaces 210 and 212. When an operator depresses footpedal 74 elastic frames 34 expand in the main axis and the added expansion of all amplified piezoelectric actuators 30(1) to 30(n) appears at coupling 40 and is transmitted to guillotine 44 in a way that cutting border 15 traverses vitrectomy port 14 exerting a footpedal controlled proportional cutting action.

Releasing footpedal 74 reverses the voltage change with a consequential contraction of amplified piezoelectric actuators because of expansion of individual piezoelectric elements 36 within frames 34. The contraction of amplified piezoelectric actuators 30 pulls guillotine mechanism 44 proximally opening vitrectomy sideport 14 by proximal displacement of cutting border 15. Tight tolerances in the mechanical elements including coupling 40 and the implementation of closed loop servo controlled circuits allow minimum backlash and linear operation.

Repetitive Mode:

In repetitive mode of operation, a cyclic voltage fluctuation (AC) is provided by the piezoelectric driver module in response to activation of footpedal 74. In this mode handpiece 10 can perform a repetitive cutting action, common in vitrectomy procedures, using a vitrectomy head 17. Typically an output frequency is produced by piezoelectric driver module, and delivered through connector 78, cables 20 and 21 to piezoelectric elements 36. Periodic voltage fluctuations produce contraction and expansion cycles of piezoelectric elements 36 in a way that elastic frames 34 are deformed producing alternating increase and reduction in the distance between surfaces 210 and 212.

The summed dimensional fluctuation of all amplified piezoelectric actuators 30(1) to 30(n) appears at coupling 40 and is transmitted in a push-pull manner to guillotine 44 in a way that cutting border 15 traverses vitrectomy port 14 in a proper amount typically above 500 microns performing a cyclic cutting action. Vacuum is usually applied at terminal 18 to enhance the cutting effect at sideport 14. FIG. 5 illustrates an array of amplified piezoelectric actuators 30 responding to a voltage signal adjusted to produce a contraction of the axial dimension of the array. Coupling element 40 and guillotine body 44 are proximally displaced in a way that cutting border 15 is proximal to vitrectomy sideport 14 leaving the sideport in an open configuration.

FIG. 6 illustrates the same array of amplified piezoelectric actuators 30 here responding to a voltage signal adjusted to produce an expansion of the axial dimension of the array. Coupling element 40 and guillotine body 44 are distally displaced in a way that cutting border 15 is distal to vitrectomy sideport 14 leaving the sideport in a closed configuration. In repetitive mode of operation, displacement of footpedal 74 activates the piezoelectric actuator driver to provide an output frequency suitable to produce cycles of motion of the cutting guillotine 15 or other scissors terminals. Cutting frequency in cuts per minute is one parameter that can be set. The sideport open-to-close ratio (duty cycle) can be regulated to maximize flow and produce the most continuous and smooth cutting action, reducing vitreous traction and the risk of damaging the retina by accidental capture.

The waveform of the axial displacement of guillotine 44 can vary between square, sine-wave, triangular, saw-tooth or any other suitable waveform for vitreous cutting. The waveform of these cycles can be factory or operator adjusted to enhance the vitreous cutting process. The assembled piezoelectric actuator handpiece 10 including detachable head 17 carrying a vitrectomy probe 12 has a characteristic resonant frequency. When operating at resonant frequency the driving voltage provided by the actuator drive module has to be reduced to about 8% of the maximum allowable voltage to compensate for the increased stroke observed at resonance that may damage the piezoelectric actuators and other mechanical components.

In repetitive mode of operation it can be desirable to run the system at resonance frequency, particularly when high speed of operation is desired. The waveform at resonant frequency is a sinusoid, having a fixed 50% duty cycle. The introduction of a proximal offset of cutting border 15 of guillotine 44 with respect to vitrectomy sideport 14 can provide a reduction of the closed duty cycle below 50% maximizing efficiency. Footpedal depression can activate the repetitive cutting action at a steady rate and simultaneously increase flow rate or vacuum. Also, a mode can exist where the cutting rate varies according to the position of the footpedal.

Vitrectomy Sideport Size Adjustment:

The piezoelectric actuator driver module can be configured using the user interface at console level to provide a baseline DC level that displaces the axial position of guillotine 44 inside probe 12 in a way that the maximally open position of cutting border 15 partially occludes sideport 14. In this way the aspirated volume of vitreous can be regulated according to proximity of delicate tissues such as the retina by changing the size of the sideport

### DESCRIPTION AND OPERATION OF ALTERNATIVE EMBODIMENTS

Active Vibration Canceling System:

One alternative embodiment shown in FIGS. 7 and 8 considers the incorporation of an accelerometer 82 properly placed to detect vibration at least in the main axis of handpiece 10. Accelerometer 82 is connected to an accelerometer module inside console 70 vitrectomy handpiece controller system through a cable 83, cable 20 and connector 78. In this configuration the array of amplified piezoelectric actuators 30 is fixed at its fixed end to the distal side of an actuator support 100 this support in rigid connection with handpiece body 16. An amplified piezoelectric actuator 84 is fixed by its fixed end to the opposite proximal side of actuator support 100. Actuator 84 movable end has an attached mass 86. Actuator 84 is connected to a vibration canceling module inside console 70 surgical handpiece controller system through a cable 85, cable 20 and connector 78. During operation actuator 84 axially displaces mass 85 up to an extent equal to distances x2−x1 to produce a vibration canceling effect according to a vibration canceling algorithm implemented at actuator controller module.

Telescopic Piezoelectric Actuators:

In the embodiment shown in FIGS. 9A, 9B, 10 and 11 a different arquitecture for a leveraged piezoelectric actuator known as telescopic piezoelectric actuators is implemented. These actuators have been characterized by Vendlinski, J and Brei, D in the paper “Dynamic behavior of telescopic actuators”, Journal of intelligent material systems and structures, p 577-585, Vol. 14, September 2003, and by Alexander, P and Brei, D in the paper “Piezoceramic telescopic actuator quasi-static experimental characterization”, Journal of intelligent material systems and structures, p 643-655, Vol. 14, October 2003.

Telescopic piezoelectric actuators are preferably constructed by creating an array of concentrically disposed piezoelectric actuators with alternating polarities. The applied voltage produces expansion of evenly disposed layers 60, 62, 64 while producing contraction of oddly disposed layers 170, 172 and vice versa. The terminal ends of neighbor concentric piezoelectric elements are bridged in alternating pairs by their terminal ends to unite expanding elements with contracting elements. In this configuration the total axial displacement of the array is the sum of the absolute displacement of each piezoelectric element in the array. One telescopic actuator having n elements will produce a displacement equal to ABS(dPZ(1))+ABS(dPZ(2))+ . . . + ABS(dPZ(n-1))+ABS(dPZ(n)), being ABS(dPZ(x)) the absolute displacement of that particular piezoelectric element.

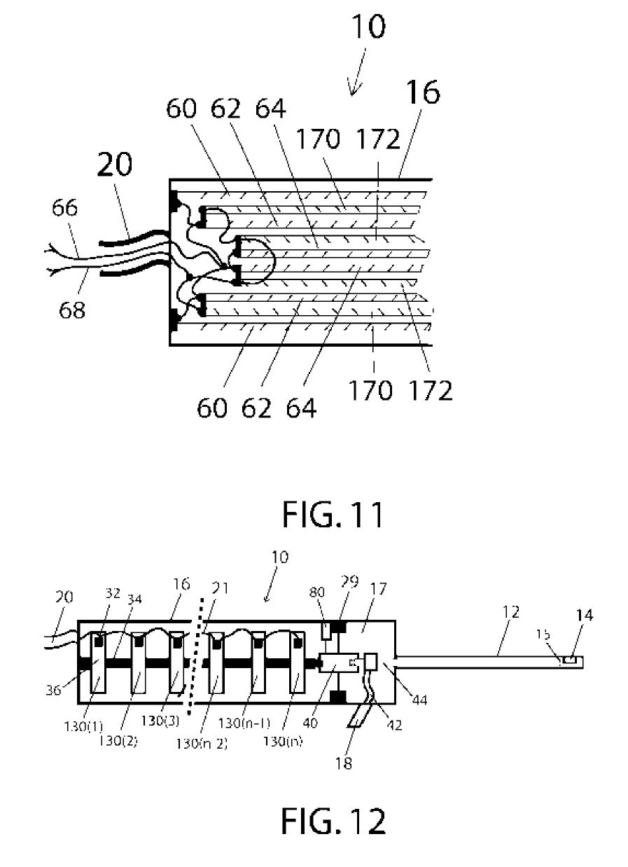

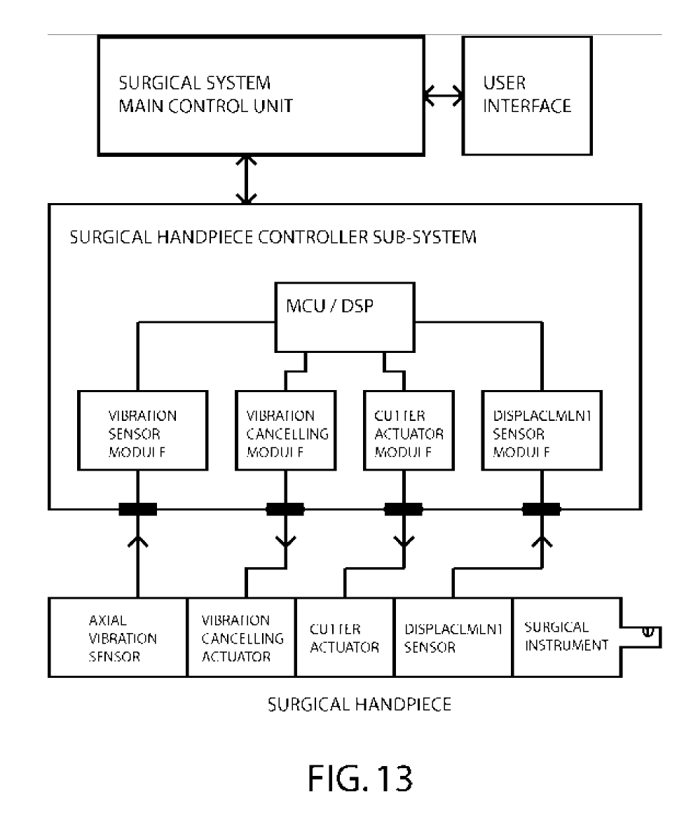

Depending on the stroke requirements for a particular surgical instrument used in conjunction with the present invention, a serially disposed array of telescopic piezoelectric actuators can provide advantageous system static, quasi-static and dynamic properties. During operation, a single telescopic piezoelectric actuator or an array of serially disposed telescopic actuators is energized in pairs to produce the summed expansion or contraction of the full array. As shown in FIG. 11 this can consider connecting in reverse polarities cables 66 and 68 to alternating piezoelectric elements of the telescopic actuator. The driving signal is provided by a cutter actuator module within a surgical handpiece controller module inside a surgical console 70. Control, feedback, vibration canceling and modes of operation are similar to that described for the main embodiment.

Bimorph Disk Actuators:

In still another embodiment the stack of amplified piezoelectric actuators 30 is replaced by other architecture for axial leveraging configured by a stack of serially coupled bimorph piezoelectric actuators, preferably in the shape of disk translators similar to model P-288 from Physik Instrumente (http://www.pi.com). The driving signal is provided by a handpiece actuator control module within a surgical handpiece controller module inside surgical console 70. Control, feedback, vibration canceling and modes of operation are similar to those described for the main embodiment.

Actuator Motion Sensor:

As depicted in FIGS. 7 and 12 surgical handpiece 10 of the present invention incorporates a piezoelectric actuator system and a surgical instrument within a detachable head (17). Handpiece body 16 can also incorporate an actuator axial displacement sensor 80 connected to a displacement sensor module that feeds a properly conditioned signal to a microcontroller or DSP in the surgical handpiece controller system inside a surgical system 70. In this way the system can operate in a closed loop servo control configuration to compensate ringing and drift conditions.

Vibration Sensor:

Handpiece body 16 can also incorporate an axial vibration sensor element typically in the form of an accelerometer 82 connected to an accelerometer sensor module that feeds a properly conditioned signal to a microcontroller or DSP in the surgical handpiece controller system inside a surgical system 70. MEMS based or other small format accelerometer ICs are suitable for this purpose. Handpiece body 16 can also incorporate a vibration canceling actuator 84 connected to a vibration canceling actuator driver module that provides the proper actuator driving signal as determined by the microcontroller or DSP in the surgical handpiece controller system inside a surgical system 70 after processing accelerometer 82 and/or displacement sensor 80 information using a vibration canceling algorithm. Also, vibration canceling actuator 84 can be activated using factory programmed vibration information stored in ROM.

### CONCLUSION, RAMIFICATIONS AND SCOPE

Thus the reader will understand that the surgical system of the invention improves over the prior art by providing a surgical handpiece that incorporates a powering method based on leveraged piezoelectric actuators. The introduction of leveraged piezoelectric actuators for the operation of the handpiece allows high speed of operation with adjustable duty cycle independent of the cut rate settings improving outflow, particularly when using vitrectomy probes of reduced diameter (23 G or less).

Using this method of activation, cut rate can be speeded up above the current limit of 1.500 cuts per minute while maintaining a high open-to-closed sideport ratio (duty cycle). By providing a biasing direct current (DC) voltage to the piezoelectric actuators, the cutting border of the guillotine can be positioned in a way that the sideport is partially occluded in the maximally open position of the guillotine.

In this way flow into the sideport is reduced limiting traction and risks of damaging delicate tissues such as the retina. The option to operate amplified piezoelectric actuators 30 in direct current (DC) mode allows an operator to control the stroke of the guillotine in proportion with the travel of an analog footpedal or other proportional user interface inputs. In this way, the cutter can be used in scissors mode, proportionally traveling along the vitrectomy sideport following the displacement of the footpedal plate.

The linear oscillatory action of the leveraged piezoelectric actuators and the mechanical simplicity of the design provide a handpiece that is silent to operate, with reduced actuator induced vibration and is lightweight. Leveraged piezoelectric actuators are also incorporated with advantage in the surgical system of this invention because of their low failure rates, reduced wear and resistance to sterilization. The short response time of the actuators allows increased open-to-close sideport ratios (duty cycle) even at high cutting rates.

The DC nature of response of the driving actuators allows adjustment of the maximum size of the effective aspiration window by providing different biasing DC voltages. A user can displace the surgical instrument in proportion to the displacement of a footpedal to operate a vitrectomy cutter, scissors or forceps in proportional mode.

Using selected driving waveforms, the handpiece operates in a vibration free, noiseless manner. While the above description provides many specificities these should not be construed as limitations on the scope of the invention, but rather as exemplifications of preferred embodiments. For example, the series of amplified piezoelectric actuators, telescopic actuators or bimorph disk translators can be replaced by other architectures of leveraged piezoelectric actuators according to stroke, force and dynamic requirements for a particular system without departing from the scope of the present invention.

The amount, dimensions, force and other static, dynamic and electric characteristics of the piezoelectric elements used can vary without departing from the scope of the present invention. The leveraged actuators can be mounted in reverse manner inverting the push-pull operation with respect to the expansion-contraction of the actuators. The relative direction of cutting border can vary from axial to oblique or rotary by changing the coupling mechanism or edge angle without departing from the scope of the present invention.

Activation of the handpiece can be made using a footpedal, sensors in the handpiece or other suitable surgical instrument operator activation method. The controller of the handpiece can be located within the same handpiece using microelectronic circuits instead of a console located controller. The controller of the handpiece can be electrically connected to the leveraged actuators in parallel or series configuration. Also, each actuator can be driven using separate output channels of the piezoelectric driver module.

The vitreous cutter head can be replaced by other linear actuator powered instruments such as scissors and forceps. The surgical handpiece can be used in other surgical procedures requiring step-mode or oscillatory activation. The probe head can be detachable or permanently assembled to the handpiece body. Accordingly, the scope of the present invention should be determined not by the embodiments illustrated but by the appended claims and their legal equivalents.

What is claimed is:

1. A precision surgical system, comprising: a) a surgical handpiece controller system, b) a surgical handpiece connected to said surgical handpiece controller system, c) said surgical handpiece including a surgical instrument actuated by linear kinetic energy, d) at least one leveraged piezoelectric actuator mechanically coupled to operate in series and mounted in said surgical handpiece for producing kinetic energy, e) means for controllably coupling said kinetic energy produced by said leveraged piezoelectric actuators to said surgical instrument, whereby said leveraged piezoelectric actuators activated by said surgical handpiece controller system provide the force and stroke to operate said surgical instrument.

2. The surgical instrument of claim 1 being a guillotine based vitrectomy probe.

3. The surgical instrument of claim 1 being a scissors.

4. The surgical instrument of claim 1 being a forceps.

5. The leveraged piezoelectric actuators of claim 1 being amplified piezoelectric actuators.

6. The leveraged piezoelectric actuators of claim 1 being telescopic piezoelectric actuators.

7. The leveraged piezoelectric actuators of claim 1 being bimorph disk translator piezoelectric actuators.

8. The surgical instrument of claim 1 being operable in repetitive mode.

9. The surgical instrument of claim 1 being operable in direct mode with motion being a function of a user interface analog input.

10. The surgical instrument of claim 1 being operable in non-resonant mode.

11. The surgical instrument of claim 1 being operable in resonant mode.

12. The surgical handpiece of claim 1 further including sensor means to detect the linear displacement produced by the leveraged piezoelectric actuators.

13. The surgical system of claim 1 being operable in closed-loop servo control modality.

14. The surgical handpiece of claim 1 further including axial vibration detection means.

15. The surgical handpiece of claim 1 further including active axial vibration canceling means.

16. The surgical system of claim 1 further including an active vibration canceling system.

17. The guillotine based vitrectomy probe of claim 2 having independently adjustable duty cycle and cut rate.

18. The guillotine based vitrectomy probe of claim 2 having an electrically adjustable area of the maximally open sideport.

19. The guillotine based vitrectomy probe of claim 2 having a plurality of operator selectable cutter displacement waveforms.

20. A method for activating a surgical instrument comprising the use of at least one leveraged piezoelectric actuator mechanically coupled to operate in series.

21. The surgical instrument of claim 20 including a vitrectomy probe.

22. The leveraged piezoelectric actuators of claim 20 being amplified piezoelectric actuators.
